(19) United States
(12) Patent Application Publication
(10) Pub. No.: US 2006/0159651 A1
(43) Pub. Date: Jul. 20, 2006

(54) Gradient molecular weight viscoelastic solutions

(76) Inventor: Michael Colvard, Encino, CA (US)

Correspondence Address: KOPPEL, PATRICK & HEYBL, 555 ST. CHARLES DRIVE, SUITE 107, THOUSAND OAKS, CA, 91360, US

(21) Appl. No.: 11/036,283

(22) Filed: Jan. 14, 2005

(51) Int. Cl. A61K 31/765; A61K 38/05; A61K 31/70

(52) U.S. Cl. 424/78.37; 514/18; 514/23

(57) ABSTRACT

A viscoelectric solution for use in a cataract removal and IOL insertion procedure comprises, in a single applicator, a liquid composition of a solution of a compound which has a range of molecular weights from about 500,000 to about 3,800,000 daltons. The applicator is filled with the compound arranged so that predominately lower molecular weight material is first delivered, higher molecular weight material is last delivered and there exists an increasing continuum of molecular weight material there between for delivery during the surgical procedure.

This application is directed to a viscoelastic solution for use in ophthalmic surgery which includes, in a single applicator, a liquid composition of a physiologically acceptable compound comprising a series of molecules of different molecular weight of said compound arranged in a gradient manner to provide both dispersive and cohesive properties at the appropriate time in a cataract lens removal and IOL placement procedure.

### BACKGROUND

Ophthalmic viscoelastic devices (OVDs), also referred to in the ophthalmic industry of ophthalmic viscosurgery devices, are widely used in ophthalmic surgery, especially during cataract extraction and intraocular lens implantation. OVD comprise viscous or viscoelastic solutions of organic molecules, depending on the compound, having molecular weights from about 25,000 to 5 million daltons, in physiological solvents.

A cataract is a cloudiness of the crystalline lens of the eye. In order to restore visual quality caused by cataract formation, standard surgical practice today involves removal of the cataract, followed by the implantation of a clear artificial intraocular lens. Anatomically, a cataract is comprised of a hard central core of dense material called the nucleus, which is surrounded by a shell of softer nuclear material called the epinucleus. The epinucleus is surrounded by a very soft shell of material called the cortex. The nucleus, epinucleus and cortex are contained within a clear cellophane-like membrane called the lens capsule or capsular bag. Circumferentially, the capsular bag is connected to the zonular fibers which are attached to the ciliary body which provides the support for the entire crystalline lens structure within the eye. (FIG. 1) Modem cataract surgery techniques involve an extracapsular cataract extraction which consists of removing the contents of the capsular bag either manually, or by a variety of mechanical means, including ultrasonic energy (phacoemulsification), laser energy and thermal energy.

OVDs are thick liquids used in modern cataract surgery to coat and protect delicate intraocular structures within the eye from collateral injury during the removal of the harder and potentially more damaging parts of the cataract material, especially the nucleus, to maintain intraocular volume and prevent collapse of the eye during surgery, to allow for better visualization and safer manipulation of instruments and devices within the eye during surgery, to lubricate the surface of an intraocular lenses prior to insertion into the eye and the inside of a lens injector used to insert a folded soft intraocular lens into the eye, and to inflate and create a space to facilitate precise placement of an intraocular lens within the capsular bag or in the area between the posterior aspect of the iris and the capsular bag, called the ciliary sulcus.

The two types of OVDs presently in clinical use are primarily dispersive or are primarily cohesive in their clinical behavior. Dispersive agents, such as VISCOAT™ (Alcon), which is a mixture of sodium hyaluronate and chondroitin sulfate, and VITRAX™ sodium hyaluronate (AMO) tend to be more protective of intraocular structures than are cohesive agents because they primarily as a coating in clinical use. The coating of intraocular structures helps to prevent damage to these structures during the process of cataract removal. Typically the nucleus is the first part of the cataract material to be removed. Damage to intraocular tissues is most likely to occur when large fragments of dense nuclear material can strike internal structures such as the corneal endothelial lining and the iris. Damage to these structures can result in loss of clarity and painful swelling of the cornea and iris, as well as post operative intraocular inflammation. This coating property of dispersive OVDs is very advantageous during the early, more turbulent and potentially more traumatic parts of the cataract procedure. The coating properties of the dispersive viscoelastics agents become progressively less necessary for tissue protection as the procedure progresses and only smaller fragments of nuclear material and much softer epinuclear and cortical material remain to be removed.

The coating qualities of dispersive agents become a disadvantage when removal of the OVD becomes necessary at the conclusion of the procedure. This is because the dispersive OVDs are more difficult to remove from the eye than are the cohesive agents. Removal of an OVD is desirable at the end of an intraocular procedure because retained OVD tends to result in an elevation of intraocular pressure post operatively. Intraocular pressure elevations occur with retained OVD because the viscoelastic material tends to clog the normal out-flow of aqueous fluid from the eye. The severity and the duration of intraocular pressure elevation is a function of the volume, concentration and molecular weight of the retained OVD. Severe or prolonged intraocular pressure can result in damage of the optic nerve, retina, iris and cornea.

Cohesive agents, such as the HEALON® products, HEALON Regular™ and HEALON GV™ (AMO), COEASE™ (AMO) and PROVISC™ (Alcon), all of which contain various different molecular weights of sodium hyaluronate or hyaluronic acid, are not as effective in coating intraocular structures as the dispersive agents and, therefore, are not as protective of intraocular tissues, but they tend to be better at retaining the spatial relationships of tissues during surgery and they are more easily aspirated and irrigated out of the eye at the end of surgery. Hyaluronic acid or its sodium salt (sodium hyaluronate) is a polysaccharide that can be up to 5 million Daltons in molecular weight. It is a linear polymer comprising up to about 12000 disaccharide units linked together with Beta-1-4 glycoside bonds. The disaccharide units are sodium glucoronate linked to an N-acetyglucosamine. These units are in turn linked together by a Beta-1-3 glucosidic bonds.

Surgeons generally prefer an agent with dispersive properties during the turbulent and potentially traumatic process of nucleus removal, because this type of agent tends to coat and protect intraocular surfaces. However, they generally tend to prefer agents with cohesive properties after the removal of the nucleus, especially during intraocular lens insertion, to maintain anterior chamber volume, lubricate the intraocular lens and to fill the capsular bag and/or the ciliary sulcus, because this type of agent is easier to remove at the end of the procedure.

Prior to the present invention, surgeons had to make a choice when performing cataract surgery and intraocular lens implantation. They can chose to use a dispersive OVD, if they want the highest level of intraocular protection, or they can chose to use a cohesive OVD, if they want the most reliable level of viscoelastic removal. An alternative is set forth in U.S. Pat. No. 5,492,936 to Francese et al which discloses methods for removing a lens from the eye which incorporates a homogeneous mixture of two different molecular weight fractions of sodium hyaluronate, namely a 2 million to 4 million molecular weight fraction and a 400,000 to 700,000 molecular weight fraction, where the mixture has the beneficial properties (as well as the negative properties) of each of the different molecular weight ranges throughout the delivery procedure. Many surgeons today chose to use both types of agents sequentially. U.S. Pat. 5,273,056 McLaughlin et al discloses a method of conducting cataract surgery using a first viscoelastic agent during the capsulotomy portion of the procedure followed sequentially by a second viscoelastic material for completion of the procedure where the two agents have different cohesive and adhesive properties. More specifically, the first viscoelastic agent is a mixture of sodium hyaluronate and chondroitin sulfate and the second agent is sodium hyaluronate Although the use of two different OVDs increases the cost of the procedure, surgeons often chose to use a dispersive viscoelastic agent early in the case when the risk of damage to intraocular structures is highest, and then, to use a cohesive agent after the denser portion of the cataract has been removed to maintain intraocular volume, to allow for better visualization, to inflate the capsular bag or the ciliary sulcus, to lubricate the intraocular lens injector and to facilitate intraocular lens insertion and placement within the capsular bag or ciliary sulcus.

The difference in the characteristics of the two basic kinds of OVD agents is related to molecular chain length, which results in different molecular weights, and concentration. The dispersive agents, such as VISCOAT™ (Alcon) and VITRAX™ (AMO) are liquid compositions with a lower molecular weight, due to shorter molecular chain length, and a higher concentration of viscoelastic material. For example, VISCOAT™ (Alcon) is a combination of sodium chondroitin sulfate at approximately 25,000 daltons and sodium hyaluronate at approximately 500,00 daltons present in concentrations of 40 mg/ml and 30 mg/ml, respectively. The cohesive agents, HEALON REGULAR™, HEALON GV™, and COEASE™ (AMO), and PROVISC™ (Alcon) are liquid compositions with a higher molecular weight due to longer molecular chain lengths and a lower concentration of viscoelastic material. For example, HEALON has a molecular weight and concentration of about 3,800,000 daltons and 10 mg/ml respectively. HEALON GV has a molecular weight of about 5,000,000 daltons at a concentration of 14 mg/ml. COEASE has a molecular weight of greater than about 1,000,000 daltons and a concentration of 12 mg/ml., and PROVISC has a molecular weight of about 2,000,000 daltons at a concentration of 10 mg/ml

To explain the dispersive versus cohesive properties of short-chained versus long-chained OVDs, a “pasta” analogy is often used by clinicians. Long-chained OVDs behave like spaghetti. The long-chained molecules of these cohesive agents become entangled and intertwined during use. When the clinician begins to aspirate the material and engages one portion of the volume of a long-chained OVD, because the molecules are intertwined the entire bulk of material tends to follow into the aspiration port. Short-chained dispersive OVDs behave more like macaroni. Removal is made more difficult, because the short-chained molecules do not tend to become intertwined. Complete removal of a short-chained OVD is difficult because only small portions of the OVD can the irrigated or aspirated with each passage of the irrigation aspiration instrument over or through the OVD.

It has also been established that the “retentiveness” of an OVD within the eye is influenced by the concentration of the viscoelastic agent. More specifically, an OVD of a give molecular weight becomes less “runny” if it is more concentrated, and more “runny”, if it is less concentrated. Increasing the concentration of a given viscoelastic agent increases the “retentiveness” or “zero-shear” viscosity of the OVD by increasing intermolecular entanglements. As a result, a material stays in place and does not tend to flow out of the eye when there is no fluid movement. This is a useful feature during certain stages of the cataract procedure. Decreasing the molecular weight and/or the concentration of a given viscoelastic agent decreases the viscosity of the OVD at zero-shear by reducing this entanglement. (Poyer J F, Chan K Y, Arshinoff S A. “Quantitative method to determine the cohesion of viscoelastic agents by dynamic aspiration”. J Cataract Refract Surg 1998: 24:1130-1135). Increasing the zero-shear viscosity of an OVD helps to increase the retentiveness of the OVD within the eye during surgery when there is no fluid movement (low shear conditions) in the eye. High zero-shear viscosity and more cohesive behavior, allows the product to be more easily removed from the eye when the OVD is being moved. With higher levels of fluid movement, for instance, during aspiration and irrigation of the viscoelastic agent, OVDs with greater molecular entanglement tend to move out of the eye more readily in a bolus-like manner. OVDs with less molecular entanglement are more difficult to aspirate. At higher levels of fluid movement, low zero-shear viscosity and dispersive behavior make a given product more likely to be retained in the eye. High zero-shear viscosity and more cohesive behavior, allows the product to be more easily removed from the eye.

### SUMMARY

An ophthalmic viscosurgery device (OVD) is provided in a delivery device, such as a syringe, where the OVD composition comprises a variety of molecular weight molecules therein, the different molecular weight molecules being arranged in the syringe in a gradient of molecular weights from lower molecular weights to higher molecular weights along the length of the syringe, the distribution of molecular weights along the length of the syringe approximating a straight line. This allows the ophthalmic surgeon to deliver, during a cataract removal and IOL placement procedure, from a single syringe, an OVD with the preferred cohesive, dispersive and retentive behavior desired for the different stages of the procedure. This may also be accomplished by providing a gradient of concentrations of the same molecular weight material along the length of the syringe, or a gradient of both molecular weights and concentrations along the syringe length.

### BRIEF DESCRIPTION OF DRAWINGS

FIG. 1 is a cross-sectional drawing showing the anatomy of a eye.

FIGS. 2-4 are cross-sectional drawings of the eye at the start of a lens removal procedure showing placement of amounts of a progressively varying OVD material into the anterior chamber of the eye.

FIGS. 5 is a cross-sectional drawing of the eye showing a device removing the capsule to expose the cataract lens.

FIGS. 6-8 are cross-sectional drawings of the eye showing removal of the lens from the capsular bag.

FIG. 9 is a cross-sectional drawing of the eye showing the partially collapsed anterior and posterior chamber.

FIG. 10 is a cross-sectional drawing of the eye showing placement of higher molecular weight OVD in the capsular bag.

FIG. 11 is a cross-sectional drawing of the eye showing an intra ocular lens (IOL) residing in the capsular bag.

FIG. 12 is a cross-sectional drawing of the eye showing an intra ocular lens (IOL) during aspiration of the OVD in the capsular bag.

FIG. 13 is a cross-sectional drawing of the eye showing removal of some OVD material from the anterior capsule.

FIG. 14 is a graphical representation of the molecular weight distribution of an OVD composition incorporating features of the invention with a molecular weights gradient as it would exist in a syringe.

FIG. 15 is a cutaway view of a syringe filled with a gradient OVD incorporating features of the invention.

### DESCRIPTION

OVD compositions incorporating features of the invention exhibit a full range of the desirable clinical characteristics, from highly dispersive to highly cohesive, in a serial manner rather than simultaneously while being disbursed from a single vial containing the material. The OVD comprises material that has a gradient of molecular weights in a single vial or syringe with the lowest molecular weight molecules of the material located in the front of the syringe or vial, so that it can be injected into the eye first, followed by molecules of the same materials of increasing or higher molecular weight further back in the syringe or vial, so that it can be injected into the eye later in the procedure. Alternatively, a gradient of OVD material in a solvent can be provided with a material of higher concentration, and therefore greatest retentiveness (i.e., zero shear viscosity), in the front of the syringe or vial, with the concentration decreases through the length of the syringe so that the least retentive material is in the back of the vial and is delivered last. A combination of these features may also be produced with a gradient ranging from the lowest molecular weight and the highest concentration to progressively higher molecular weights at lower concentrations.

A variety of physiologically acceptable solvents can be used to dissolve or dilute the OVD. Balanced Salt Solution (BSS) is the intraocular irrigating solution used universally during cataract surgery to keep the eye inflated, to help in the removal of lens material and to maintain normal pressure and volume relationships of the eye. The aqueous fluid of the eye normally contains sodium bicarbonate buffers, glutathione and glucose. Studies have shown that the addition of bicarbonate buffers, glutathione and glucose to standard Balanced Salt Solution helps to reduce endothelial dysfunction and reduce the risks of post operative corneal swelling. (Glasser D. G., Matsuda M., Ellis, J. G., Edelhauser, H. F., Effects of Intraocular Solutions on the Corneal Endothelium After In Vivo Anterior Chamber Irrigation. Amer. Journal of Phth. 1985: 99:321-328) Following cataract surgery, some material OVD, particularly OVD material of low molecular weight, will likely remain in the eye until it is gradually cleared by the trabecular meshwork. Therefore, the addition of sodium bicarbonate buffers, glutathione and glucose to an OVD which incorporates features of the invention is likely to provide a similar beneficial effect on the physiologic stability and function of the corneal endothelium. Preferably, the OVD solution also has a pH and osmolality similar to or substantially the same as the aqueous humor of the eye. Therefore, a preferred solvent is a balanced salt solution (BSS), such as commonly used in ophthalmic procedure, which is also supplemented with compounds shown to maintain the endothelial function of tissue surfaces within the eye. Preferably, glutathione, glucose and bicarbonate buffer, so that the OVD/solvent combination has a pH and osmolality similar to that of the aqueous humor. Other additives which may also be added to the OVD/BSS solution include phosphate, lactate and ascorbate containing salts. A particularly preferred solvent is an aqueous solution containing, on a mmol/liter basis, about 160 mmol of sodium, 5 mmol of potassium, 1 mmol of calcium, 1.0 mmol of magnesium, 130 mmol of chloride, 25 mmol of bicarbonate, 3 mmol of phosphate, 5 mmol of glucose and 0.3 mmol of glutathione. The preferred osmolality (which can be obtained by varying the constituents of the BSS or the concentration of the OVD) is 305, or slightly greater, to match the osmolality of the cornea.

The invention contemplates providing a syringe 100 of OVD material 10 adequate for a complete cataract procedure. While the invention includes use of a single chemical entity with a range of distinctly different properties along the length of a single syringe 100 for serial delivery, a preferred embodiment comprises providing an OVD material 10 with a gradient of properties preferred for the complete procedure, with the intermediate section 102 of the syringe 12 (the center of the syringe 100) having that portion of the OVD, i.e., the intermediate molecular weight or concentration OVD 102, with properties intermediate between those of the OVD 104, 106 portion at either end of the syringe 12.

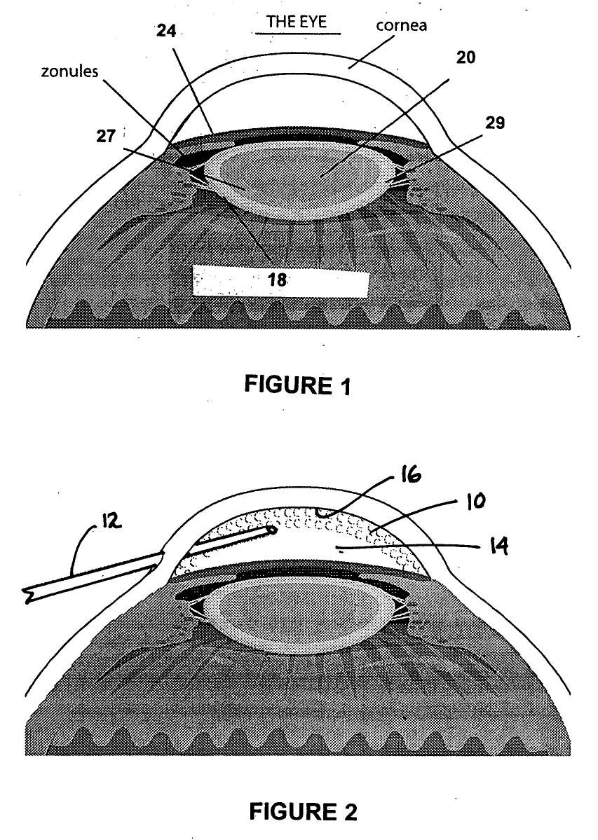

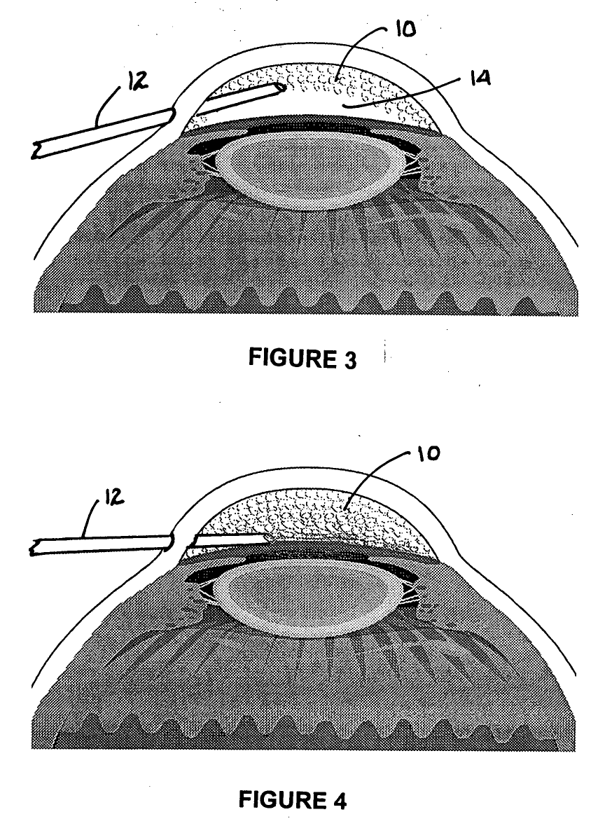

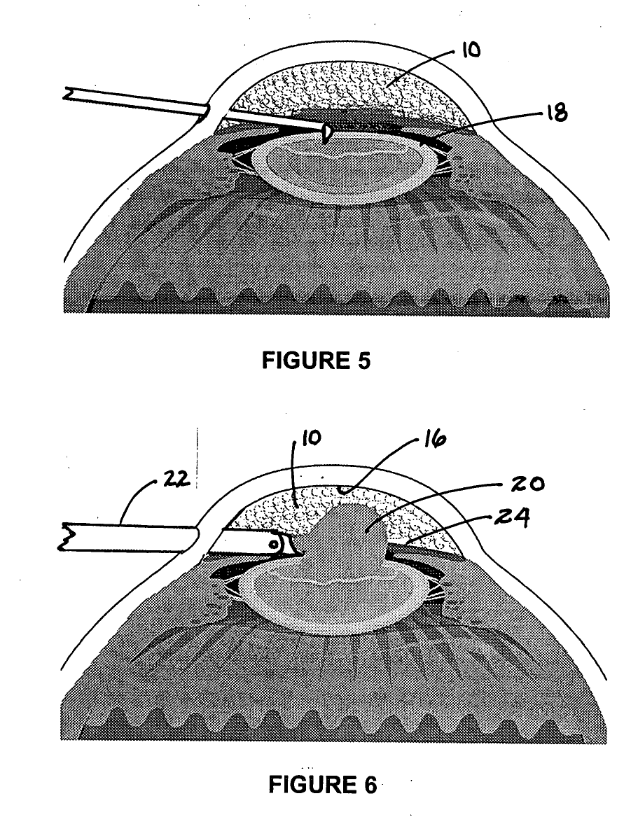

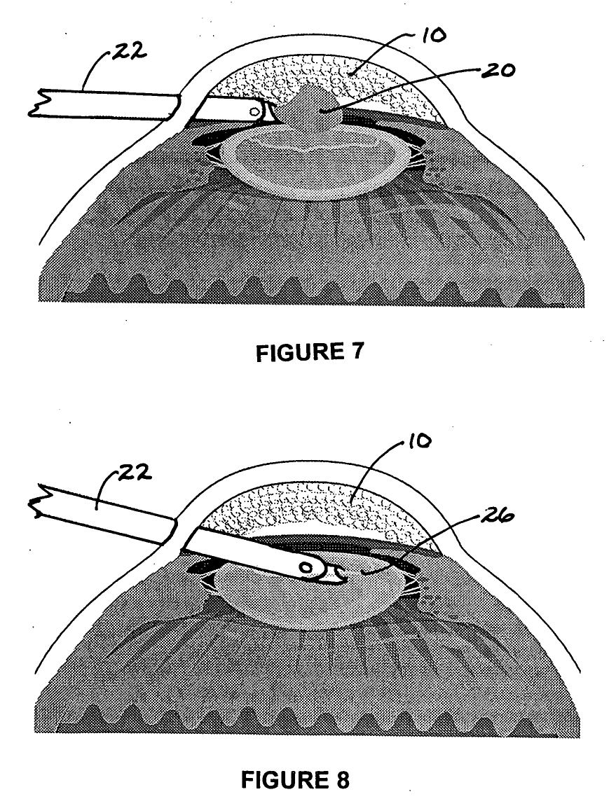

With reference to FIG. 1-13, the use of an OVD 10, incorporating features of the invention, in a cataract removal and IOL placement procedure, is described. That portion of the ophthalmic viscoelastic material with the lowest molecular weight 104, and, therefore, the most dispersive and most protective in terms of its coating qualities is positioned in the syringe 100 so it is injected first. This allows placement of the most protective OVD 10 material against the comeal endothelium for maximal protection. As additional amounts of the OVD is added from the same syringe 100 through a delivery tube 12 to the eye to fill the anterior chamber 14 of the eye due to the gradient of molecular weight material within the OVD 10, higher molecular weight material is provided so that a very thin layer of the very lowest molecular weight 104 material is placed against the comeal endothelium 16 followed by layers of material of progressively higher molecular weight 102, 106 (FIGS. 2-4). The capsule 18 is then opened (FIG. 5), and a cannula 22 is used to remove the nucleus 20 (FIGS. 6-7). During the removal of the nucleus 20, some of the OVD 10 is aspirated along with the fragments of dense nuclear material. The OVD 10 of progressively longer molecular chains (higher molecular weight) is more likely to be aspirated during this process, while the shorter chained, more coating OVD 10 is less likely to be aspirated. The gradient OVD 10 composition provides the more dispersive material portion 104 against the corneal endothelium 16 and iris 24, where it is most needed. The further injection of progressively higher molecular weigh OVD 102 into the anterior chamber 14 insures that the added material, while still more protective than a uniformly cohesive OVD, will be more likely to leave the eye during nucleus 20 removal as well as easier to remove at the conclusion of the procedure than a uniformly dispersive OVD.

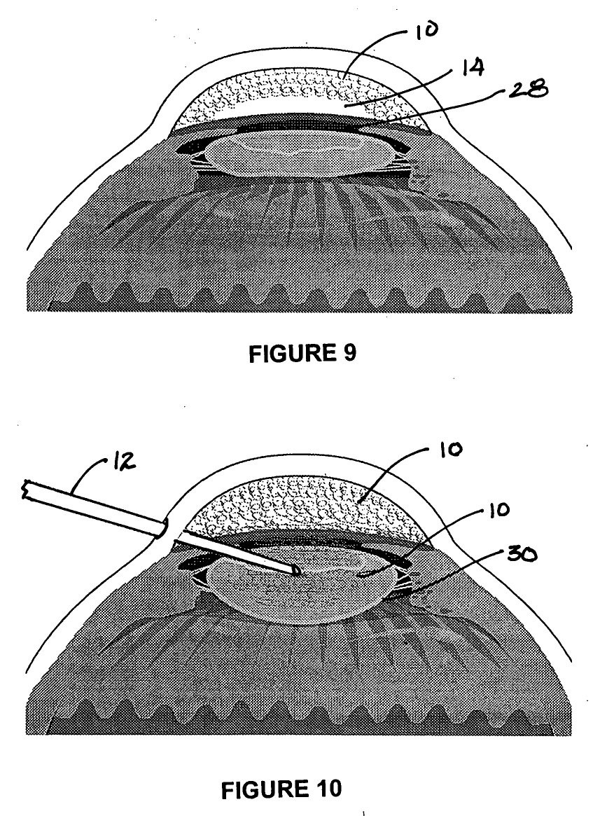

As remaining cataract material 26 in the eye becomes progressively less, both in terms of density and volume, the need from the more dispersive, highly protective material diminishes (FIG. 8). After all of the cataract material 26, including softer epinucleus 27 and cortex 29, has been removed, the cataract extraction instruments have been withdrawn from the eye, and irrigation fluid is no longer flowing into the eye, the anterior chamber 14 and the posterior chamber 28 of the eye lose volume and partially collapse (FIG. 9). Additional OVD material of a higher molecular weight 106 is placed in the eye, replacing lower molecular weight OVD 10 material 102, 104 which has been aspirated or which has flowed out of the eye during cataract removal.

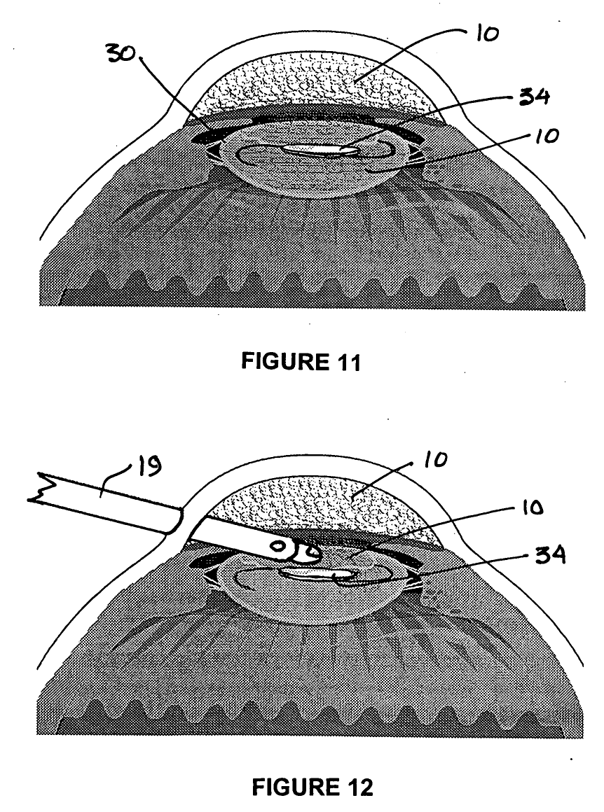

Continued delivery of more OVD 10 provides a more cohesive, progressively longer chained OVD material 106, refilling first the anterior chamber 14, then the anterior portion of the capsular bag 30 and finally the most posterior portion of the capsular bag 30. The portion of the gradient OVD material 10 having the highest molecular weight with most cohesive properties 106 (the last delivered material) is then placed at the bottom of the capsular bag 30 (FIG. 10). A small amount of the most cohesive material 106 is also used to lubricate the intraocular lens injector (not shown). The intraocular lens 34 is placed within the capsular bag 30 or the ciliary sulcus (FIG. 11 shows capsular bag placement). The OVD 10 remaining in the eye is then aspirated. The most cohesive, longest chained portion 106 of the OVD 10 is inside the capsular bag 30 and under the IOL 34. Although the OVD 10 beneath the IOL 34 is the most difficult to access, the high molecular weight of this material facilitates aspiration (FIG. 12). The gradient OVD material 10 above the intraocular lens 34, which is easy to access, is still very cohesive and easy to remove.

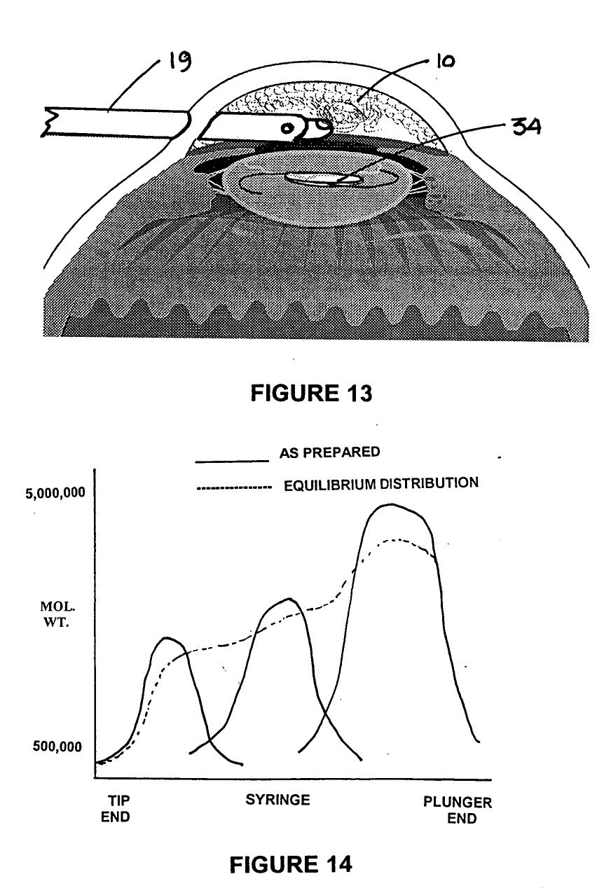

A thin layer of the most difficult to remove, lowest molecular weight OVD 104 may still remain as a coating against the corneal endothelium 16 (FIG. 13). However, the total amount of the progressive gradient OVD 10 remaining in the eye is much smaller than the amount of OVD 10 which would have remained if only an OVD of low molecular weight had been used. The level of protection afforded by the gradient OVD 10 is superior to the level of protection which would have been provided if only an OVD of higher molecular weight had been used. Only a single syringe 100 of gradient OVD 10 material, not separate syringes of different OVD material, is needed to provide this full range of desired viscoelastic properties.

While the procedure and composition set forth above describes the use of lower and higher molecular weight material, as indicated above, in each instance a gradient of higher and lower concentrations can be used.

An OVD material incorporating features of the invention comprises various molecular weights or concentrations of hyaluronic acid or sodium hyaluronate from about 25,000 daltons, but preferably 200,000 daltons (the lower molecular weight material) to about 5,000,000 daltons (the higher molecular weight material) and more preferably from about 500,000 to 3,800,000 daltons. Alternatively, hyaluronic acid or Na hyaluronate in gradient concentrations from about 50 mg/ml to about 5 mg/ml may be employed. Where a gradient of concentrations is used it is preferred to use diluted fractions of a higher molecular weight material, for example from about 1 to 2 million daltons, with the molecular weight chosen so that the highest concentration material has the intended cohesive properties during the later stages of the procedure. Even more flexibility or range in cohesive and dispersive properties of the OVD can be obtained by forming the gradient by providing, in a single delivery instrument a gradient of properties provided by high concentrations of lower molecular weight materials to lower concentrations of higher molecular weight materials. Also the concentration of the lower molecular weight material can be increased to increase osmolality and zero shear viscosity. However, depending on the molecular weight of the OVD used, it may also be beneficial to reverse the concentration gradient.

In a typical prior practiced cataract removal and lens insertion procedure, a surgeon would use approximately 0.4-1.0 cc of OVD materials, which may be provided by multiple syringes each containing a different OVD material. In contrast, the preferred OVD material 10, incorporating features of the invention, comprises a single syringe 100 containing 0.4-1.0 cc of hyaluronic acid or sodium hyaluronate with a gradient of molecular weights with the lower molecular weight starting at about 25,000 daltons, but preferably at about 200,000 to 5 million daltons, more preferably 500,000-3,800,000, with the lower molecular weight molecules 104 located in the tip end of the syringe and the higher molecular weight molecules 106 located in the plunger end of the syringe with the gradient of molecular weight approximating a straight line curve between the lower molecular weight and the higher weight. However, larger volumes of OVD may be packaged in a single syringe. Still further the concentration of the OVD may be constant along the length of the syringe or range from about 5 mg/ml to about 50 mg/ml. An advantage of increasing concentration, as discussed above, is that it results in increased osmolality. Further, increasing the concentration of an OVD encourages greater entanglement of molecular chains which in turn increases its viscosity at zero shear (i.e., greater retentiveness when there is little flow). Increasing the concentration of the very low molecular weight dispersive portion 104 of sodium hyaluronate has the advantage of increasing the retentiveness, thus improving chamber maintenance during capsulorhexis and the increase in the osmolality of that portion of the OVD which is most likely to remain in contact with the endothelium after surgery will improve the post-op corneal clarity. In addition, reducing the concentration of the higher molecular weight material would reduce the risks of elevated intraocular pressure post-operatively.

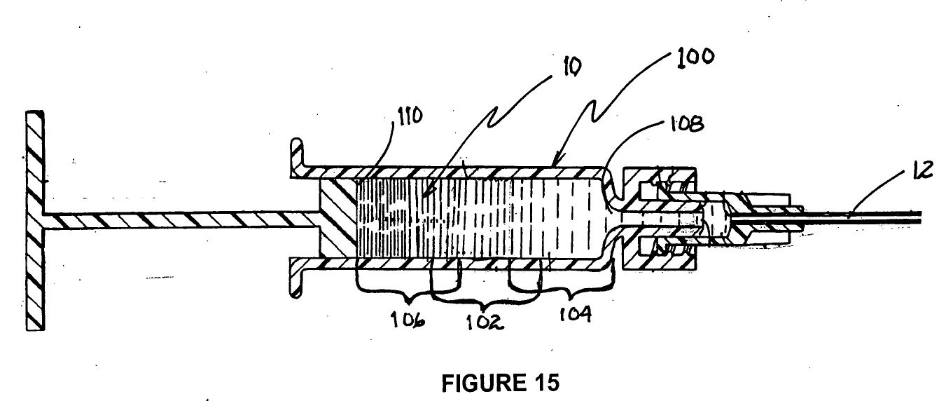

Many OVD materials, and particularly hyaluronic acid and sodium hyaluronate, are now available as a composition having a discrete or narrow distribution of molecular weights. The composition generally contains a bell curve distribution of different molecular weights and when a molecular weight is specified it is typically an average molecular weight. The gradient OVD described herein can be generated by assembling, in a single syringe, layered amounts of the same OVD material having various identified molecular weight compositions, from lower to higher molecular weight from the tip or delivery end 108 of the syringe 100 to the plunger end 110 of the syringe 100. Normal diffusion will then create an overlapping of the bell curve distributions of the different molecular weight materials such as shown in FIGS. 14 and 15 to create a straight line distribution of molecular weights for illustrative purposes. However, complete diffusion does not occur and the composition tends to generally retain regions of different but progressively greater molecular weight approximating a straight line relationship of increasing molecular weight. FIG. 14 illustrates a composition prepared from three different molecular weight OVD to create the desired single composition, represented by the dashed line, with an increasing molecular weight gradient approximating a sloped straight line distribution. However, the intended gradient can be obtained by arranging more than 3 OVD compositions or, to a lesser degree using only 2 different OVD compositions. FIG. 15 shows the gradient OVD 10 enclosed in a syringe 100. The syringe is merely representative of a delivery device which may be used and one skilled in the art will recognize that numerous syringe designs previously available or available in the future may be used to hold and deliver the OVD 10. This increased density of vertical lines within the internal volume of the syringe is intended to demonstrate the increasing molecular weight of the OVD along the length of the syringe 100 with the forward portion containing (i.e., the delivery end 108) containing predominantly the lower molecular weight OVD fraction 104, the rearward portion (i.e., the plunger end 110) containing the higher molecular weight OVD fraction 106 and the intermediate portion containing an intermediate molecular weight portion 102, the intermediate portion 102 overlapping each of the higher molecular weight 106 and lower molecular weight 102 portions to provide a continuous gradient of molecular weight throughout the length of the syringe 100.

As a result, instead of sequentially introducing into the surgical site multiple viscous or viscoelastic agents with different cohesive and adherent properties, the surgeon can now provide a single viscous and viscoelastic material with a continuum of varying physical properties necessary for each stage of the lens removal and/or placement procedure. As an alternative, normal diffusion gel filtration or permeation chromatography can also be used to separate molecules of a single hyaluronic acid OVD according to their size (molecular weight). In practice a column is filled with highly porous beads, the dimensions of the pores chosen to select a certain molecular size for separation. Examples of such column materials include cross-linked hydrophilic polystyrene, porous glass, SephadexR, SephacrylR, and SepharoseR gels. These column materials can separate a homogeneous mixture of hyaluronic acid or sodium hyaluronate into many fractions each having very narrow but different molecular weight ranges. These many fractions can then be stacked in a syringe as described above rearranging the molecules of a single composition into a gradient with continuously varying viscous (i.e., cohesive, dispersive, and retentive) properties.

One skilled in the art will recognize that, based on the teachings herein, in light of the various different OVD materials available for ophthalmic procedures, the invention can be practiced using other OVD compounds arranged in a similar manner. As an example, the gradients can be prepared by arranging different molecular weight portions, or providing increasing concentrations or combinations of different molecular weights and concentrations of hyaluronic acid, sodium hyaluronate, chondroitin sulfate, polyacrylamide, hydroxypropylmethylcellulose, proteoglycans, collagen, methylcellulose, carboxymethylcellulose, ethylcellulose and keratin. As a further alternative, the OVD gradient can be created using a blend of different OVD materials rather than just different concentrations or different molecular weights of the same materials. Also, while it is preferred to use a single syringe of a gradient of molecular weight material, the objective of the invention may be accomplished by dividing the gradient of molecular weight material into two or more syringes, used serially in order of increasing molecular weight.

I claim:

1. A liquid composition having both dispersive and cohesive properties for use in an ophthalmic procedures comprising a material composed of molecules with a range of molecular weights in a physiologically acceptable solvent, said liquid composition contained in a dispenser with the molecules of the material arranged in a molecular weight gradient with lower molecular weight molecules located in a first portion of the dispenser for first delivery, higher molecular weight molecules located in a rear portion of the dispenser for last delivery and molecules with intermediate molecular weights located in the dispenser for delivery after the lower molecular weight material but before the higher molecular weight material.

2. The composition of claim 1 wherein the lower molecular weight material in the first portion is present in a ratio to solvent in said first portion greater than the ratio to solvent in the final portion, of the higher molecular weight material.

3. The composition of claim 1 wherein the range of molecular weights is from about 200,000 Daltons to about 5 million Daltons.

4. The composition of claim 3 wherein the molecular weight gradient along the length of the dispenser from the first portion to the last portion is substantially a straight line distribution from about 200,000 Daltons to about 5 million Dalton.

5. The composition of claim 1 wherein the range of molecular weights is from about 500,000 Dalton to about 3.8 million Dalton.

6. The composition of claim 5 wherein the molecular weight gradient along the length of the dispenser from the first portion to the last portion is substantially a straight line distribution from about 500,000 Dalton to about 3.8 million Dalton.

7. The composition of claim 1 wherein the dispenser is a syringe with a capacity of from about 0.4 cc to about 1.0 cc of the liquid composition.

8. The composition of claim 1 wherein the solvent is a balanced salt solution with pH similar to that of the aqueous humor and an osmolality similar to that of the cornea.

9. The composition of claim 1 wherein the solvent contains one or more of glutathione, glucose and bicarbonate buffer.

10. An improved method for performing a cataract removal and intraocular lens insertion procedure utilizing a liquid viscosurgical device to coat or protect tissue surfaces, lubricate surgical devices and maintain ophthalmic volume wherein the liquid viscosurgical device is delivered from a single syringe holding a sufficient quantity of said liquid viscosurgical device for the complete procedure, said liquid viscosurgical device first delivered from the syringe having predominantly dispersive properties and the liquid viscosurgical device last delivered from the syringe having predominantly cohesive properties.

11. The improved method of claim 10 wherein the liquid viscosurgical device in the syringe comprises molecules of different molecular weights arranged in an aqueous solvent in a gradient from molecules of lower molecular weight positioned for first delivery to molecules of highest molecular weight positioned for last delivery.

12. The improved method of claim 11 wherein the concentration of the lower molecular weight molecules first delivered is greater than the concentration of molecules of highest molecular weight last delivered.

13. The improved method of claim 10 wherein the liquid viscosurgical device in the syringe comprises molecules of substantially the same molecular weight arranged in a gradient of molecules present in a lower concentration for first delivery to molecules present in a higher concentration for last delivery.

14. The method of claim 11 wherein the range of molecular weights is from about 200,000 Daltons to about 5 million Daltons.

15. The method of claim 11 wherein a gradient of molecular weights from the first delivered molecules to the last delivered molecules is substantially a straight line distribution from about 200,000 Dalton to about 5 million Dalton.

16. The method of claim 1 wherein the range of molecular weights is from about 200,000 Daltons to about 5 million Daltons.

17. The method of claim 11 wherein a gradient of molecular weights from the first delivered molecules to the last delivered molecules is substantially a straight line distribution from about 200,000 Dalton to about 5 million Dalton.

18. The composition of claim 11 wherein the aqueous solvent is a balanced salt solution with pH similar to that of the aqueous humor and an osmolality similar to that of the cornea.

19. The composition of claim 18 wherein the solvent contains one or more of glutathione, glucose and bicarbonate buffer.

20. A liquid composition having both dispersive and cohesive properties for use in an ophthalmic procedures to coat or protect tissue surfaces, lubricate surgical devices and maintain ophthalmic volume comprising a liquid viscosurgical device in an aqueous solvent contained in a delivery syringe wherein the liquid viscosurgical device has a substantially uniform molecular weight distribution along the length of the syringe and a gradient of properties from predominantly dispersive properties at a dispensing end of the syringe to predominantly cohesive properties at a plunger end of the syringe, said gradient of properties provided by an increasing concentration from a lower concentration of the viscosurgical device in the solvent at the dispensing end to a higher concentration of the viscosurgical device in the solvent at the plunger end of the syringe.

21. The composition of claim 20 wherein the average molecular weight of the viscosurgical device is about 2 million Daltons and the concentration varies from about 5 mg/ml at the dispensing end to about 50 mg/ml at the plunger end of the syringe.

22. The composition of claim 20 wherein the aqueous solvent is a balanced salt solution with pH similar to that of the aqueous humor and an osmolality similar to that of the cornea.

23. The composition of claim 22 wherein the solvent contains one or more of glutathione, glucose and bicarbonate buffer.

24. The improved method of claim 10 wherein the liquid viscosurgical device in the syringe comprises molecules of substantially the same molecular weight arranged in a gradient of molecules present in a higher concentration for first delivery to molecules present in a lower concentration for last delivery.
